(19) United States
(12) Patent Application Publication
(10) Pub. No.: US 2023/0126306 A1
(43) Pub. Date: Apr. 27, 2023

(54) MATERIAL DETERIORATION EVALUATION DEVICE AND MATERIAL DETERIORATION EVALUATION METHOD

(71) Applicant: TOSHIBA ENERGY SYSTEMS & SOLUTIONS CORPORATION, Kawasaki-shi (JP)

(72) Inventors: Tetsuya SHITAKA, Yokohama (JP); Yusuke SUZUKI, Yokohama (JP); Keisuke TAKAKI, Yokohama (JP); Yoshikazu NINOMIYA, Nerima (JP); Tsukasa TAKEUCHI, Yokohama (JP); Yasuteru KAWAI, Yokohama (JP)

(73) Assignee: TOSHIBA ENERGY SYSTEMS & SOLUTIONS CORPORATION, Kawasaki-shi (JP)

(21) Appl. No.: 17/867,793

(22) Filed: Jul. 19, 2022

(30) Foreign Application Priority Data
Oct. 26, 2021 (JP) 2021-174758

(51) Int. Cl. G01N 25/72; G01N 33/2045

(52) U.S. Cl. CPC G01N 25/72; G01N 33/2045

(57) ABSTRACT

A material deterioration evaluation device for evaluating embrittlement of equipment, includes: an operation data obtaining unit that detects and obtains a state of the equipment as operation data; an operation data storage unit that saves the operation data; a temperature evaluation unit that calculates a predetermined evaluation-target site temperature of the equipment based on the operation data; an evaluation-target component material storage unit that stores material data of a material forming the equipment and embrittlement estimation formulas; an embrittlement evaluation unit that calculates an embrittlement quantity of the material forming the equipment based on the evaluation-target site temperature, the material data, and the embrittlement estimation formulas; a risk evaluation unit that calculates a damage risk of the material that forms the equipment based on the embrittlement quantity; and a recommended maintenance time presentation unit that presents a recommended maintenance time of the equipment based on the damage risk.

### CROSS REFERENCE TO RELATED APPLICATION

This application is based upon and claims the benefit of priority from Japanese Patent Application No. 2021-174758, filed on Oct. 26, 2021; the entire contents of which are incorporated herein by reference.

### FIELD

Embodiments disclosed herein relate to a material deterioration evaluation device and a material deterioration evaluation method.

### BACKGROUND

Materials used in high-temperature environments are known to undergo material deterioration over time. For example, turbines, casings, control valves, piping, and the like, which are major components of thermal power plants, are exposed to high-temperature environments due to incoming steam, which causes material deterioration such as softening and embrittlement over time. Since such material deterioration is related to strength properties such as tensile strength and yield strength of a member, it is necessary to quantitatively understand a material deterioration quantity and conduct maintenance and management such as deterioration evaluation and component replacement at appropriate timing to properly evaluate damage and service life associated with equipment operation.

Embrittlement, a form of material deterioration, is a factor in brittle fracture of equipment. Brittle fracture is a phenomenon in which cracks propagate rapidly and lead to fracture, almost without plastic deformation. For example, a turbine shaft rotating at high speed in a high-temperature environment (hereinafter referred to simply as “rotor”) is subjected to centrifugal stress in the rotor during operation. The embrittlement of the rotor material reduces its toughness, and in some cases, the centrifugal stress has led to brittle fracture.

Embrittlement not only causes such brittle fracture but also affects crack growth properties. Crack growth is known to be caused by creep and metal fatigue. Creep is a phenomenon in which crack initiation, growth, and rupture occur as a result of gradual permanent deformation of a metal material over time, even at low stress below the metal material's yield strength when the metal material is used in an environment at about half its melting point. Fatigue is a phenomenon in which cracks are initiated and grow due to repeated exposure to stresses that do not cause rupture under static load, leading to failure.

One of these damage evaluation methods is to predict and evaluate a quantity of crack growth, and to evaluate a damage quantity in a member based on an allowable crack length. A crack growth rate can be evaluated in advance by material strength tests, but the crack growth rate is affected by material embrittlement and the crack growth rate increases with material embrittlement. The allowable crack length also decreases with material embrittlement. Therefore, the evaluation of crack growth damage due to creep and fatigue should be combined with the evaluation of the material embrittlement quantity.

It is known that use temperature, operating time, and impurity element quantities contained in the material affect the embrittlement of the material. Rotors and turbine casings are examples of equipment for which embrittlement evaluation is important. The rotor is a rotary shaft that transmits rotational force received by rotor blades from a steam flow to a power generator, and the turbine casing is a covering that surrounds the rotor. Steam flows between the turbine casing and the rotor, and the rotor blades provided on a rotor's outer periphery obtain the rotational force from the steam flow. Multiple stages of rotor blades are arranged on the rotor's outer periphery, and these rotor blades receive the steam flow, which generates rotational force in the rotor. On the other hand, steam that flows in at a high temperature consumes energy by passing through each stage of the rotor blades, and steam temperature decreases as the steam flows downstream. Therefore, the rotor and turbine casing in contact with the steam are used in a high-temperature environment, and temperature distribution also occurs within the same member. This temperature distribution causes different degrees of embrittlement in different sites of the same member.

Conventional thermal power generation is mainly based on base-load operation and is often operated near rated power to maximize plant efficiency. In this type of operation, the temperature and pressure in each site of the rotor and turbine casing are very clear because they are carefully evaluated and optimized at the time of turbine design, and they do not vary much during operation. Therefore, it has been relatively easy to determine a transition of an embrittlement quantity by opening the turbine during plant stop and conducting periodic embrittlement evaluation.

However, in recent years, with the spread of renewable energy, there are more and more opportunities to conduct a part-load operation in thermal power stations. The increase in part-load operation increases the number of operations outside a design point, exposing the turbine equipment to temperatures that were not anticipated at the time of design for a long time. As mentioned above, embrittlement is affected by the use temperature, and the embrittlement rate also changes with variations in the use temperature, making it difficult to properly determine the transition of the embrittlement quantity by only periodically measuring embrittlement during plant stop as in the past.

In addition, stopping the power plant and opening the steam turbine increase costs of power generation due to time and effort involved. Therefore, it is difficult to ensure the sufficient number of embrittlement evaluations due to cost constraints.

Therefore, there is a need for an embrittlement evaluation method and evaluation device that do not involve stopping and opening the equipment, but also take into account temperature changes during equipment operation. Although there have been cases in which embrittlement prediction of turbine rotor materials has been studied based on use temperature, time, and material data, there are many cases in which embrittlement in a range of about 300 to 450° C. has been verified. Applicability of these methods and devices to actual equipment, which requires evaluation over a wider temperature range, is unknown. Depending on the material, there have been cases where the embrittlement quantity peaked at around 300 to 400° C. when the material was isothermally held at a certain temperature. However, when combined with crack growth, for example, the higher the temperature, the faster the crack growth rate, and crack growth evaluation in the temperature range above 500° C. is also expected in creep crack growth. In other words, a damage evaluation-target site is not necessarily a maximum embrittled site, and an embrittlement prediction method that can be applied to a wider temperature range of 500° C. or higher is needed.

A damage evaluation device that collects and uses operation data, or other data, and combines material deterioration evaluation such as embrittlement has already been proposed. In this device, an embrittlement quantity is estimated using a prediction formula previously obtained from experimental data. However, it is widely known that there are variations in material properties, and embrittlement is no exception. That is, statistical evaluation that takes variation into account is necessary also for the embrittlement estimation. To improve accuracy of statistical evaluation, a large quantity of experimental data is required, but it is time-consuming to previously obtain a sufficient quantity of experimental data on embrittlement that progresses over time. However, no method or device configuration has been developed to utilize newly obtained embrittlement data to improve the accuracy of estimation in conventional damage evaluation devices and embrittlement evaluation methods.

Although turbine rotors and casings being thermal power equipment were used as examples in the above, embrittlement evaluation is not limited to these products or fields. It is also expected to apply the embrittlement evaluation to high-temperature members in other equipment, such as high-temperature steam piping, fuel cells, and engine components, for example.

The damage evaluation device, combined with the material deterioration evaluation, has a device configuration including sensors that obtain operation data, various arithmetic processing units, data storage units, and devices used for data input and presentation. Similarly, when a device, which predicts material deterioration, deformation, and the like based on operation data, is configured, the same processes are required for the sensors that obtain operation data and some arithmetic processing units. Therefore, when multiple evaluation devices such as the damage evaluation devices and the material deterioration evaluation devices are applied, the device configuration has duplicated functions. This is undesirable in terms of cost and space-saving.

Thus, embrittlement is a type of material deterioration that progresses over time and is affected by use temperature, operating time, and the quantities of impurity elements in the material. In recent years, power generators or the like with increased opportunities for the part-load operation has experienced variations in use temperature, making it difficult to properly predict and estimate embrittlement with conventional periodic embrittlement measurements. In addition, it is undesirable in terms of cost to conduct the sufficient number of direct measurements, with product disassembly. Methods and devices have been proposed to estimate the embrittlement quantity based on the use temperature, the operating time, and the quantity of impurity elements in the material, but their applicability over a wide temperature range is unknown. Furthermore, existing embrittlement prediction formulas and embrittlement estimation devices using these formulas make it difficult to utilize newly obtained embrittlement measurement data, so improvement in the accuracy of embrittlement estimation during a period of device operation cannot be expected. In addition, the applicability of the device, which integrates the various equipment and arithmetic processing units that configure the evaluation device, also remains issues.

The present invention was made in consideration of these conventional circumstances, and an object thereof is to provide a material deterioration evaluation device and a material deterioration evaluation method that can respond to diversified equipment operations and achieve highly reliable evaluation.

### BRIEF DESCRIPTION OF THE DRAWINGS

FIG. 1 is a block diagram illustrating a configuration of a material deterioration evaluation device according to a first embodiment.

FIG. 2 is a schematic diagram illustrating an example of installation positions of sensors according to the first embodiment.

FIG. 3 is a flowchart illustrating operation of an operation data obtaining unit according to the first embodiment.

FIG. 4 is a flowchart illustrating operation of a material embrittlement quantity evaluation unit according to the first embodiment.

FIG. 5 is a diagram illustrating a comparison between an embrittlement estimation method according to the first embodiment and an estimation method based on a known example.

FIG. 6 is a diagram illustrating an example of estimation results of the embrittlement estimation method according to the first embodiment.

FIG. 7 are diagrams each illustrating an example of verification results of embrittlement estimation accuracy according to the first embodiment.

FIG. 8 is a table illustrating operation of a risk evaluation unit according to the first embodiment.

FIG. 9 is a schematic diagram illustrating a configuration of a material deterioration evaluation device according to a second embodiment.

FIG. 10 is a schematic diagram illustrating a shared example of an evaluation unit, and other units, which configure the material deterioration evaluation device according to the second embodiment.

### DETAILED DESCRIPTION

A material deterioration evaluation device of an embodiment is a material deterioration evaluation device for evaluating embrittlement of equipment, and includes: an operation data obtaining unit that detects and obtains a state of the equipment as operation data; an operation data storage unit that stores the operation data; a temperature evaluation unit that calculates a predetermined evaluation-target site temperature of the equipment based on the operation data; an evaluation-target component material storage unit that stores material data of a material forming the equipment and embrittlement estimation formulas; an embrittlement evaluation unit that calculates an embrittlement quantity of the material forming the equipment based on the evaluation-target site temperature, the material data, and the embrittlement estimation formulas; a risk evaluation unit that calculates a damage risk of the material that forms the equipment based on the embrittlement quantity; and a recommended maintenance time presentation unit that presents a recommended maintenance time of the equipment based on the damage risk.

According to the embodiment, it is possible to provide a material deterioration evaluation device and a material deterioration evaluation method that can respond to diversified equipment operations and achieve highly reliable evaluation.

Hereinafter, a material deterioration evaluation device and a material deterioration evaluation method according to the embodiment are described with reference to the drawings.

### Configuration of First Embodiment

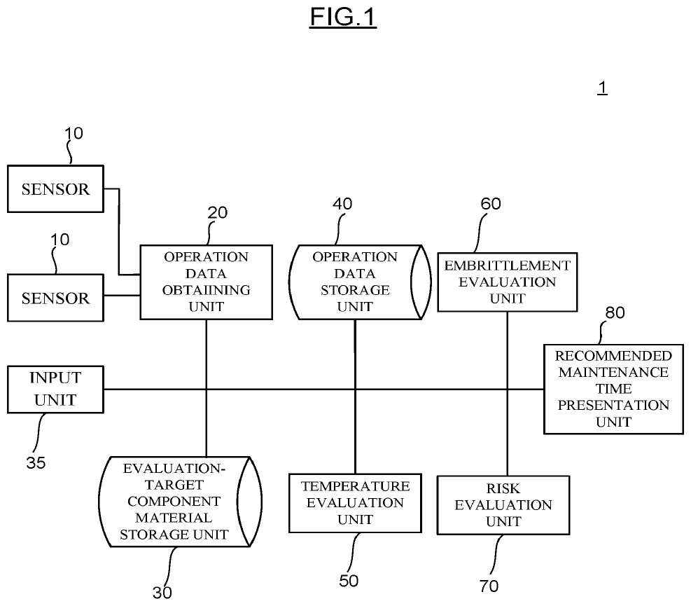

A first embodiment is described below with reference to FIG. 1. As illustrated in FIG. 1, a material deterioration evaluation device 1 of the first embodiment includes sensors 10, an operation data obtaining unit 20, an evaluation-target component material storage unit 30, an input unit 35, an operation data storage unit 40, a temperature evaluation unit 50, an embrittlement evaluation unit 60, a risk evaluation unit 70, and a recommended maintenance time presentation unit 80.

The operation data obtaining unit 20 is an arithmetic block that obtains operation data through the sensors 10. The evaluation-target component material storage unit 30 stores material data of embrittlement evaluation-target members. The input unit 35 is an input interface, such as a keyboard, for example, and is used to previously store material data and other data in the evaluation-target component material storage unit 30 and other units. The input unit 35 also has a terminal for information communication, such as a LAN port, to store data obtained from equipment outside the device in the evaluation-target component material storage unit 30 and other units. The operation data storage unit 40 stores operation data indicating a state of each site of components forming equipment to be evaluated, including the embrittlement evaluation-target members. The temperature evaluation unit 50 is an arithmetic block that evaluates an evaluation-target site temperature based on the operation data and other data obtained by the operation data obtaining unit 20. The embrittlement evaluation unit 60 is an arithmetic block that evaluates a material embrittlement quantity using the evaluation results of the temperature evaluation unit 50 and material data such as chemical components of evaluation-target components. The risk evaluation unit 70 is an arithmetic block that evaluates a failure risk based on the evaluation results of the material embrittlement quantity. The recommended maintenance time presentation unit 80 is an interface that presents a recommended maintenance time to users based on the evaluation results of the failure risk and a future plant operation plan.

The evaluation-target component material storage unit 30 stores material data such as chemical components and strength properties of evaluation-target members. The evaluation-target component material storage unit 30 may also store embrittlement evaluation results obtained from time to time. The evaluation-target component material storage unit 30 can be fabricated by a nonvolatile memory, a hard disk drive, or other devices.

The operation data storage unit 40 stores operation data indicating the state of each site of the components forming the equipment to be evaluated, including the embrittlement evaluation-target members. The operation data storage unit 40 can be fabricated by a nonvolatile memory, a hard disk drive, or other devices. In addition to storing the obtained operation data, the operation data storage unit 40 may also store past operation data before the operation of this deterioration evaluation device is started as historical data. The historical data includes the operation data obtained from time to time, total operating time and other accumulation histories, a change quantity in temperature, pressure, and the like of each site during startup and stop or power variation, a change quantity per unit time, and so on. In addition to the operation data and its historical data, the operation data storage unit 40 may also store state quantities corresponding to the operation data of the evaluation-target site and its historical data.

(Sensor 10)

The sensor 10 obtains operation data of the equipment to be evaluated, including the embrittlement evaluation-target members. Examples of the operation data obtained by the sensor 10 include, for example, temperature, pressure, strain, and the like. In addition, the sensor 10 may also detect power, a load ratio, and the like of the equipment. The sensor 10 is either pre-installed during equipment design and manufacturing or newly added for evaluation.

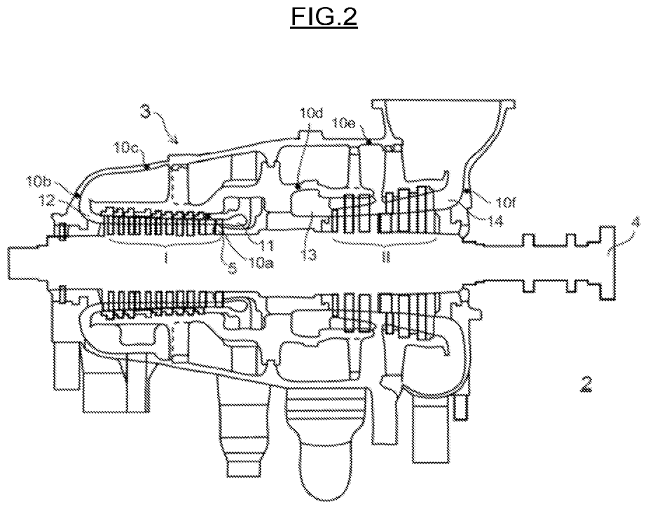

An operational example in a turbine casing is given as an application example of the sensor 10. FIG. 2 illustrates an example of installation positions of sensors in a turbine casing 3 of turbine equipment 2. The turbine equipment 2 illustrated in FIG. 2 has the turbine casing 3, a rotor 4, and a plurality of rotor blades 5 that form a stage group I and a stage group II. The turbine casing 3 is provided with sensors 10a to 10f. In the example illustrated in FIG. 2, the sensors 10a to 10f are temperature sensors that detect temperature.

The sensor 10a is arranged at an area from a steam inlet 11 to near a wake flow of the first-stage rotor blade 5 in the turbine casing 3. The sensor 10b is arranged near a steam outlet 12 of the stage group I in the turbine casing 3. The sensor 10c is arranged near a steam passage after the steam outlet 12 in the turbine casing 3. The sensor 10d is arranged near a steam inlet 13 of the stage group II in the turbine casing 3. The sensor 10e is arranged near the stage group II in the turbine casing 3. The sensor 10f is arranged near a steam outlet 14 of the stage group II in the turbine casing 3.

In the turbine casing 3 illustrated in FIG. 2, when estimating the temperature of each stage in the stage group I, detection results of the sensor 10a near the steam inlet 11 and the sensor 10b near the steam outlet 12 can be used. The sensor 10c near the steam passage after the steam outlet 12 can also be used instead of the sensor 10b. However, when estimating steam inlet and outlet temperatures, an estimation error in the steam inlet/outlet temperatures can be reduced by estimating the steam temperature from temperature measurement data at positions near the steam inlet/outlet. In the above example, it is easier to estimate the steam outlet temperature by using the sensor 10b than the sensor 10c. When estimating each stage temperature from the steam inlet/outlet temperatures, estimation accuracy can be expected to improve by selecting an appropriate sensor because the estimation accuracy of each stage temperature is affected by the estimation error in the steam inlet/outlet side temperatures.

The sensor 10a is arranged at the area from the steam inlet 11 to near the wake flow of the first-stage rotor blade 5 in the turbine casing 3, but the position is not limited thereto. The installation position of each sensor depends on design conditions and is not limited to the wake flow of the first-stage rotor blade 5, but may be at other positions. For example, when the design of the turbine casing 3 makes it difficult to measure the temperature at the position of the sensor 10b, the temperature of the steam passage part after the steam outlet 12 that can be detected by the sensor 10c may be used for estimation. A sensor may be installed between stages in the same manner as the sensor 10a.

The number of sensors 10 to extract the operation data is not limited to two locations near the steam inlet or steam outlet, as it depends on the number of sites to evaluate the state quantity, the position, and the estimation formula. For example, when the temperature is estimated from the steam inlet 11 to near the wake flow of the first-stage rotor blade 5 in the stage group I, the data of the sensor 10a close to the evaluation-target site may be sufficient. In addition, the data to be extracted for estimating the state quantity of each stage in the stage group II are the data obtained by detecting the temperature at each site by using two sensors from among the sensor 10d and sensor 10e or sensor 10f, or three sensors of the sensor 10d, sensor 10e, and sensor 10f. In addition, the sensors 10 at multiple locations may be prepared as data extraction targets, and the sensor to collect data may be selected according to the data to be collected such as steam pressure and operating power, or data to be estimated such as estimated state quantity at any given time. The same is true for pressure and strain sensors, and the arrangement of sensors can be determined according to the locations and contents of the operation data to be detected.

For estimating the temperature of each stage, the method described above uses the measured values of the temperature sensors in the vicinity of or before and after the stage to be estimated, but a measured value of a pressure sensor may be used together with the measured values of the temperature sensors. For example, when calculating heat transfer, a kinematic viscosity coefficient, Reynolds number, Nusselt number, Prandtl number, and so on can be used in the calculation. Steam pressure is also required as a parameter in the calculation of these values. In this case, the pressure sensor is installed as the sensor 10, and the values of these parameters are calculated based on the measured value of the pressure sensor and combined with the measured values of the temperature sensors. This allows the temperature of the stage to be estimated to be calculated.

(Operation Data Obtaining Unit 20)

The operation data obtaining unit 20 has functions of obtaining the operation data measured during operation by the sensors 10 installed in the turbine equipment 2 at an appropriate sampling frequency, averaging and applying denoising, and outputting the data to a post-process. In addition, the operation data obtaining unit 20 selects the sensors 10 arranged at various locations of the turbine equipment 2 according to the contents of the operation data to be obtained and obtains desired operation data from the sensors 10. In other words, when obtaining certain operation data, it is possible to set from which sensors what kind of data (temperature, pressure, and the like) is to be obtained.

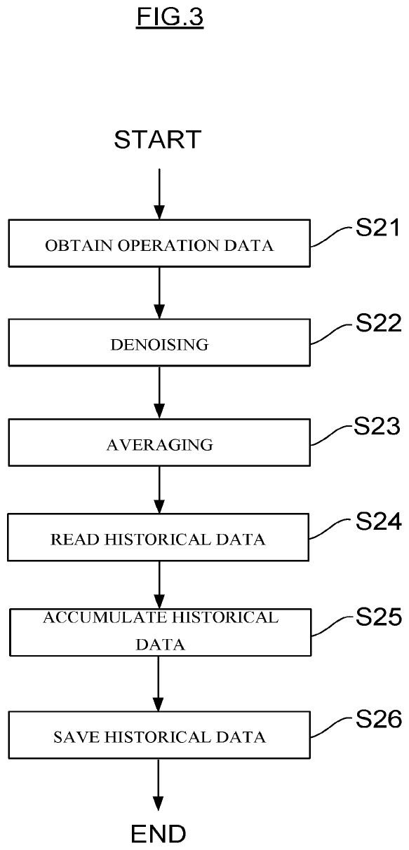

FIG. 3 illustrates obtaining operation of the operation data by the operation data obtaining unit 20. The operation data obtaining unit 20 reads detection data such as the temperature and pressure, plant power, load ratio, and other operation data from the sensors 10 installed in the turbine equipment 2 (S21).

After reading the operation data, the operation data obtaining unit 20 performs data processing such as denoising (S22) and averaging (S23) to reduce the data.

Once the operation data is reduced, the operation data obtaining unit 20 reads historical data from the operation data storage unit 40 (S24). Examples of the historical data include the operation data obtained from time to time, total operating time and other accumulation histories, a change quantity in temperature, pressure, and the like of each site during startup and stop or power variation, a change quantity per unit time, and the like. That is, the operation data obtaining unit 20 obtains the past history of the operation data in addition to the operation data obtained through the sensors 10. The historical data may further include transient data indicating a state of a plant at an evaluated time, data accumulating these transient data, and data obtained by adding or subtracting data at multiple predetermined times, such as the change quantity in temperature and pressure of each site in the turbine equipment 2. The operation data obtaining unit 20 obtains the historical data from the operation data storage unit 40.

The operation data obtaining unit 20 generates historical data by accumulating and performing arithmetic processing of the transient data (operation data) from the operation data storage unit 40 (S25). The generated historical data is saved in the operation data storage unit 40 (S26). The historical data generated by the operation data obtaining unit 20 is not limited to those based on the operation data obtained through the sensors 10. In the case of an existing plant that has been continuously operated for a certain period, the operation data obtaining unit 20 may obtain and accumulate operation history from the start of the operation to the time of installation of the device and store the operation history in the operation data storage unit 40. The operation data may be input through the input unit 35 and saved in the operation data storage unit 40.

(Temperature Evaluation Unit 50)

The temperature evaluation unit 50 calculates the temperature of a predetermined evaluation-target site using the operation data and historical data obtained and generated by the operation data obtaining unit 20. Examples of calculation methods used by the temperature evaluation unit 50 include: (1) a method of obtaining a heat balance through a balance calculation by estimating a working fluid temperature based on the temperature data of steam or the like at working fluid inlet/outlet or the like as in the turbine casing 3 illustrated in FIG. 2, operating conditions such as power of the equipment regarding the temperature at a certain evaluation-target site; (2) a method of calculating the temperature by previously creating a relational formula between the measurement data of various sensors installed at predetermined positions of the equipment to be evaluated and the evaluation-target site temperature, and other methods, for example. Here, the operating conditions such as the equipment output, the relational formula with the temperature of each site, and the like can be previously saved in the operation data storage unit 40.

Depending on a plant configuration in which the material deterioration evaluation device 1 is installed, the equipment to be evaluated, and the number of evaluation-target sites, it may be difficult to calculate and process all of the operation data that are sent sequentially. In such a case, for example, state quantities such as temperature and pressure of the evaluation-target site can be previously stored in the operation data storage unit 40 for assumed operation data, and the temperature at a predetermined evaluation-target site may be output using the state quantities stored in the operation data storage unit 40 instead of the operation data obtained by the sensors 10.

(Embrittlement Evaluation Unit 60)

The embrittlement evaluation unit 60 estimates an embrittlement quantity in any evaluation-target site of the equipment to be evaluated based on the temperature at the predetermined evaluation-target site calculated by the temperature evaluation unit 50 and the material data of an evaluation-target component previously stored in the evaluation-target component material storage unit 30. Examples of the material data include a chemical component, crystal grain size, and strength data such as hardness, yield strength, and an impact value of the material forming the member to be evaluated. The embrittlement quantity calculated sequentially in the embrittlement evaluation unit 60 is also stored in the evaluation-target component material storage unit 30, and this embrittlement quantity is also included in the material data.

The embrittlement quantity is expressed, for example, in terms of fracture appearance transition temperature (FATT).

The fracture appearance transition temperature is the temperature at which a ductile fracture ratio, which indicates a ratio of a ductile fracture surface to a brittle fracture surface, corresponds to 50% in a fracture surface obtained by an impact test. Metal materials tend to become less ductile and more brittle as temperature decreases. When a material becomes embrittled, the ductile fracture ratio of 50% is no longer exhibited unless the temperature is higher. That is, the fracture appearance transition temperature FATT increases with embrittlement. The embrittlement quantity may be expressed by using an increased quantity ΔFATTₜ between the FATT at the time of manufacture of an evaluation-target site material (FATT₀) and the FATT at the current time (FATTₜ).

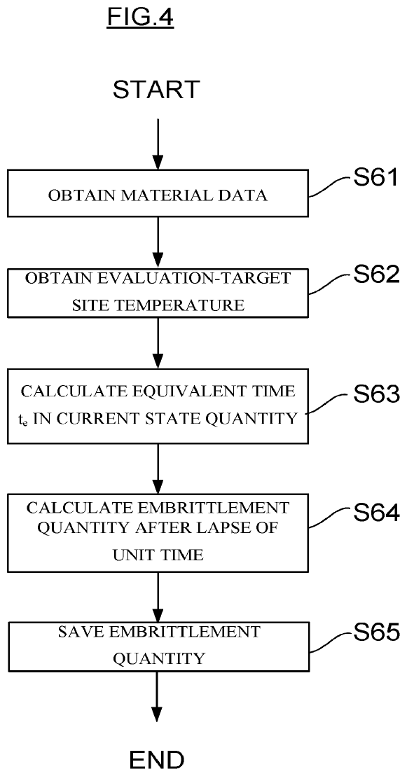

FIG. 4 presents an example of the evaluation operation by the embrittlement evaluation unit 60. The embrittlement evaluation unit 60 calculates the embrittlement quantity after the lapse of unit time. Here, the unit time refers to a certain time zone within a time in which the input evaluation-target site temperature can be considered constant. The unit time can be set in advance, or determined sequentially by monitoring changes in an operating state quantity and a change quantity in the evaluation-target site temperature, which is the input for the calculation. The embrittlement evaluation unit 60 obtains the material data of the evaluation-target component stored in the evaluation-target component material storage unit 30 (S61), then obtains the evaluation-target site temperature calculated by the temperature evaluation unit 50 (S62).

Using the obtained material data, the evaluation-target site temperature calculated by the temperature evaluation unit 50, and embrittlement estimation formulas described below, an equivalent time tₑ in the current state quantity is calculated (S63). The equivalent time tₑ refers to the time required to reach the current embrittlement quantity when the temperature at the current time is isothermally held.

After a lapse of unit time Δt, when the temperature can be regarded as constant, an apparent isothermal holding time t is calculated using the following formula (1), and the embrittlement estimation formula, material data, evaluation-target site temperature, and the above-mentioned apparent holding time t are used to calculate the embrittlement quantity after the lapse of unit time (S64).

t=tₑ+Δt (1)

Next, the embrittlement evaluation unit 60 saves the calculated embrittlement quantity in the evaluation-target component material storage unit 30 (S65).

The embrittlement evaluation unit 60 can calculate the embrittlement quantity using the following embrittlement estimation formulas, which assume that, for example, the embrittlement quantity is in proportion to a grain boundary segregation quantity of impurity elements.

(embrittlement quantity ΔFATTₜ)/(saturated embrittlement quantity ΔFATT_{∞})=(1−exp(X²)×erfc(X)) (2)

where X is a function of a constant Y calculated from the isothermal holding temperature T [° C.], the holding time t [Hr], and a predetermined element quantity in the material.

X=f(t,T,Y) (3)

Once the saturated embrittlement quantity ΔFATT_{∞} at the same state quantity is determined by using formulas (2) and (3) above, the embrittlement quantity ΔFATTₜ at a certain time can be obtained.

One feature of the embrittlement estimation method presented in this embodiment is a form of the formula expressing the saturated embrittlement quantity, which uses an exponential function whose exponent is an inverse of a linear function of temperature, as expressed in formula (4) below.

(saturated embrittlement quantity ΔFATT_{∞})=A1×B×exp{A2/(T+273)} (4)

where A1 and A2 are constants obtained experimentally and B is a constant calculated from the material data. A denominator of the exponent is represented by (T+273), but if it is the linear function of temperature, the denominator may be T+273.15 or an absolute temperature (K).

The following is a detailed explanation of the features of this embodiment in comparison with a known example. In the known example, the saturated embrittlement quantity of CrMoV steel is estimated by the following formula (5), which is combined with the above formulas (2) and (3) to estimate the embrittlement quantity.

(saturated embrittlement quantity ΔFATT_{∞})=425.0+1.778×K−0.9643×T−0.001990×K×T (5)

where K is a constant calculated from a predetermined element quantity in the material and T is a holding temperature.

Formula (5) is derived by approximating the saturated embrittlement quantity as the linear function of temperature based on embrittlement measurement data in the temperature range of 300 to 450° C. Although the accuracy of estimating the embrittlement quantity using formula (5) has been verified in the above temperature range, the estimation accuracy in the higher temperature range was unknown. Therefore, the estimation accuracy of formula (5) was verified using embrittlement measurement data of CrMoV steel after 90,000 to 140,000 hours of holding at 470 to 520° C.

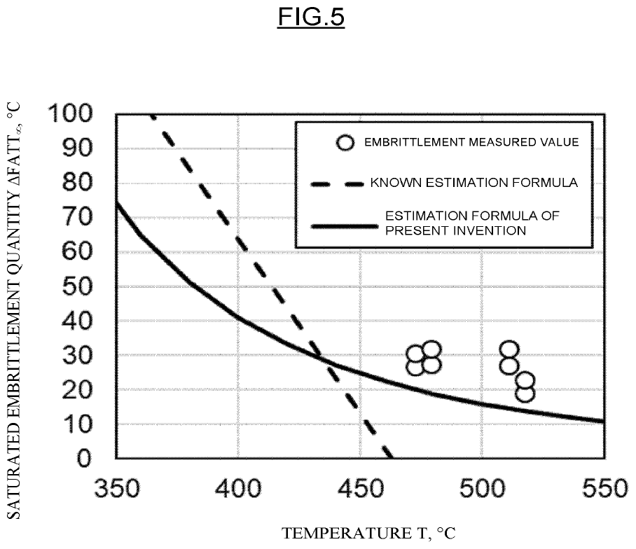

A graphic chart in FIG. 5 presents a comparison of the embrittlement estimation method between this embodiment and the known example. The graphic chart presents the relationship by placing the holding temperature (° C.) on the horizontal axis and the saturated embrittlement quantity (° C.) on the vertical axis. Plots in the chart represent the embrittlement measurement data at 470 to 520° C. and the saturated embrittlement quantity calculated using formula (4), and the dashed line represents estimation results of the saturated embrittlement quantity using formula (5) in the known example.

The estimated value of the saturated embrittlement quantity based on the known example is zero at the holding temperature of about 460° C. and results in no embrittlement in the temperature range above 460° C. However, embrittlement has been observed in CrMoV steel after holding at 470 to 520° C., which is inconsistent with the estimation results. From these results, it seems that the limit of application of the estimation based on the known example is up to the holding temperature of about 460° C., although there is an influence of the constant K calculated from the predetermined element quantity in the material, and that it is difficult to apply this method to the estimation of embrittlement including the temperature range of 450° C. or higher.

On the other hand, the solid line in the chart represents the saturated embrittlement quantity estimated by formula (4), and even in the temperature range of 450° C. or higher, the estimated value of the saturated embrittlement quantity is not zero due to the form of the formula, and there is no clear upper-temperature limit for application. The saturated embrittlement quantity in the vicinity of the holding temperature of 500° C. can be estimated with an error of about 10° C.

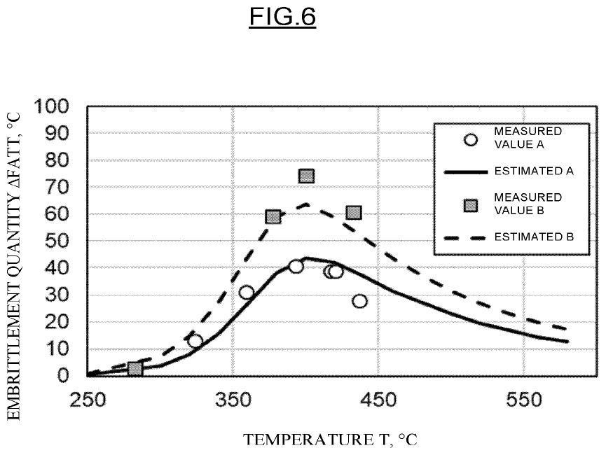

FIG. 6 presents the estimation results of the embrittlement quantity at 280 to 450° C. using formulas (4) and (2). Plots in the chart represent the embrittlement measured values, and the solid and dashed lines represent the estimation results. Measured values A and B are the embrittlement measurement results on specimens taken from the same CrMoV steel, respectively. The estimated values and measured values are in good agreement, and the embrittlement estimation using formula (4) is also applicable to temperatures at 280 to 450° C.

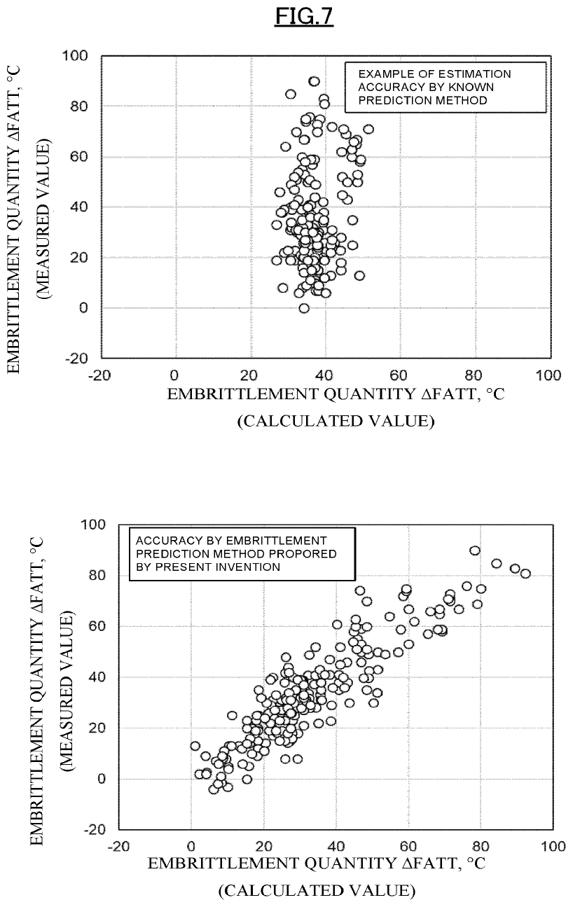

FIG. 7 present results of verifying the accuracy of embrittlement estimation using formulas (4) and (2). For comparison, results of the accuracy verification using the known example are also presented. The embrittlement measurement data used for the accuracy verification are the CrMoV steel embrittlement measurement data after 30,000 to 280,000 hours of holding at the temperature range of 280 to 536° C. The number of data is about 200, of which about 70% are the embrittlement measurement data held at the temperature of 450° C. or higher.

The upper chart presents the prediction accuracy by the known estimation method and estimation errors that are about twice as large as the measured value relative to the estimated value are observed for some data. On the other hand, the lower chart presents the results of the embrittlement estimation using formula (4). It can be seen that the estimated values and measured values are in general agreement.

These results indicate that estimating the saturated embrittlement quantity as the exponential function of the inverse of the temperature as expressed in formula (4) enables appropriate prediction of the embrittlement quantity over a wider temperature range than in the known example, and the use of this formula solves the problem of establishing the embrittlement estimation method applicable to the temperature range exceeding 500° C.

The constant B in formula (4) is a value calculated from the material data of the evaluation-target member. There is a method where weights by mass of predetermined impurity elements from the chemical component of the evaluation-target member are weighted and added together, as one example of a determination method of the constant B.

B=α·D+β·E+γ·F+δ·G (6)

where D, E, F, and G are wt % of predetermined impurity element quantities, and α, β, γ, and δ are weighting coefficients.

Formula (6) is an example of a calculation based on four impurity element quantities, but the number of elements used to calculate the constant B is not limited to four. Since a type of elements used depends on a target material, the number of terms in the above formula can be increased or decreased accordingly. The elements to be used in the coefficient calculation are selected and calculated according to the material from among eight elements of P, Si, Mn, Cu, Ni, Sn, Sb, and As as impurity elements in the case of heat-resistant steel. The constant B can also be calculated by multiplying predetermined impurity element quantities.

Depending on the equipment to be evaluated and the number of evaluation-target sites of the material deterioration evaluation device 1, it may be difficult to process all of the temperature data that are sent sequentially. In such a case, an embrittlement rate can be calculated in advance using the embrittlement estimation formulas for the material data, use temperature, and the embrittlement quantity at the current time, and the embrittlement quantity per unit time may be calculated using this embrittlement rate and added to output the embrittlement quantity at a predetermined evaluation-target site. Alternatively, part of the calculation process, such as the relationship between the right side of formula (2) and variable X, can be calculated in advance and used to measure the embrittlement quantity. In any case, one feature of the embrittlement prediction method in this embodiment is to use the saturated embrittlement quantity estimation formula that includes the exponential function whose exponent is the inverse of the linear function of temperature, as expressed in formula (4).

Depending on the member to be evaluated, there may be cases where there is no measured record of some of the impurity element quantities used to calculate the constant B, and the element quantities necessary to calculate the constant may not be available. In such cases, the impurity element quantities may be assumed. For example, an average value of the impurity element quantities of multiple similar metal members can be used. In addition, standard deviations and the like may also be used to calculate the estimated value. Such statistics may be values calculated by selecting data based on a location of manufacture, manufacturer, age of manufacture, and the like.

The embrittlement estimation formula, a standard error in the embrittlement estimation formula, the embrittlement rate for each state quantity, the statistics of the impurity element quantities, and the like can be previously stored in the evaluation-target component material storage unit 30. The constants A1 and A2 in formula (4), the standard error of embrittlement, and the statistics of various impurity element quantities are all values calculated from experimental data. When the number of experimental data used for the calculation is small, reliability of the above coefficients and statistics is also low, and the estimated values of the embrittlement quantity using these values are also not reliable. During the operation of the material deterioration evaluation device 1, embrittlement evaluations with direct measurements at the evaluation-target site, other sites, and other products are conducted, and results of these measurements and experimental data are accumulated in a database or the like outside the material deterioration evaluation device 1. By using the measurement data stored in the database, it is possible to recalculate the above coefficients and statistics such as standard errors by combining the embrittlement measurement data obtained daily with the conventional embrittlement measurement data.

In the statistics of various impurity element quantities, the statistics can be recalculated in the same way by collecting the results of element quantity measurement on other members. The reliability of the embrittlement evaluation by the material deterioration evaluation device 1 can be improved by making it possible to update the various coefficients and statistics stored in the evaluation-target component material storage unit 30 based on the results of these calculations. To achieve this, the input unit 35 is provided with a terminal for data communication to enable updating the data in the evaluation-target component material storage unit 30. The material deterioration evaluation device 1 can be put online through LAN, or the like, and the data in the evaluation-target component material storage unit 30 can be updated automatically at regular or certain times, or by using various media, and the like.

When the above coefficients and statistics are updated, the embrittlement quantity at the current time of the site to be evaluated may be reevaluated based on the historical data saved in the operation data storage unit.

The embrittlement evaluation unit 60 also has a function to back-calculate B (the constant calculated from the material data) in formula (4) from the embrittlement measurement results by the direct measurement and the operation data. As a concrete example, consider a case where the material deterioration evaluation device 1 is applied to a product after a certain period of operation. When determining the constant B from the material data as in formula (6), the element quantities in the site to be evaluated are required. When there is no material data at the time of product manufacture, it is possible to cut off a part of the product and analyze the material from the cut-off part, but in many cases, it is not acceptable to cut off the product during operation. In such cases, the constant B is back-calculated using formulas (1) to (4) above by inputting the historical data of operation from the start of the operation to the current time and the embrittlement measurement results at the current time. When there is no historical data from the start of the operation, the constant B may be back-calculated based on the embrittlement measurement results at predetermined times t1 and t2 and the historical data of operation from t1 to t2. In the example above, the minimum number of embrittlement measurements required for the back-calculation of the constant B is indicated, but multiple embrittlement measurement results may be combined to approximate the constant B therefrom. The calculated constant B is stored in the evaluation-target component material storage unit 30.

(Risk Evaluation Unit 70)

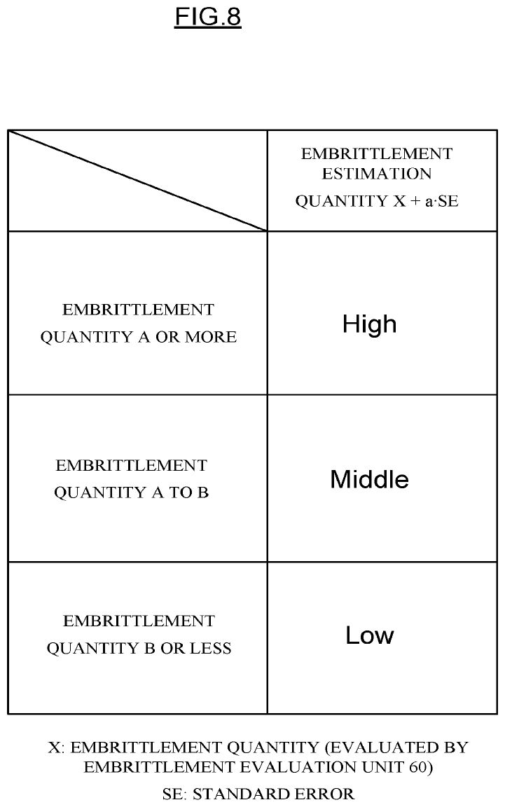

The risk evaluation unit 70 evaluates a damage risk to the evaluation-target member based on the embrittlement quantity evaluated by the embrittlement evaluation unit 60 and the statistics such as the standard error in the embrittlement estimation formula and the standard deviation stored in the evaluation-target component material storage unit 30. FIG. 8 illustrates an example of damage risk evaluation. The embrittlement quantity evaluated by the embrittlement evaluation unit 60 is combined with the standard error in the embrittlement estimation by formulas (2) to (4) to calculate an estimated value considering a confidence interval. The risk is evaluated from the estimated value and a threshold value of the embrittlement quantity. The statistics such as the standard error and standard deviation are rewritable.

Threshold values A, B, and a in the table may be values determined in advance from design conditions, and the like, or they may vary depending on the operating time of the equipment to be evaluated and the evaluation-target site temperature. The number of these threshold values is not limited to the above values. The operating time, use temperature, and so on may be added as parameters. All of the threshold values can also be stored in the evaluation-target component material memory unit 30 and updated based on information obtained from equipment, or the like outside the material deterioration evaluation device 1.

(Recommended Maintenance Time Presentation Unit 80)

The recommended maintenance time presentation unit 80 presents a recommended inspection time such as a direct measurement of embrittlement or a recommended maintenance time such as a component replacement time based on the current damage risk generated by the risk evaluation unit 70 and a future damage risk based on the operation plan. The recommended maintenance time presentation unit 80 has a display device such as a display unit and can present the contents of proposals to a user. The recommended maintenance time presentation unit 80 has a function to evaluate a damage risk in the future based on the operation plan of the equipment to be evaluated, which is separately input by the user through the input unit 35. Here, the operation plan is information indicating, for example, equipment operating rate, average power of the equipment, start and stop frequency, and the like, and is stored in advance in the operation data storage unit 40. The recommended maintenance time presentation unit 80 calculates the predicted embrittlement quantity for a given operation plan based on the obtained embrittlement quantity and the operation data and historical data associated with the embrittlement quantity.

The first embodiment has been described above. The first embodiment can reduce the number of embrittlement measurements that involve stopping and opening the equipment to be evaluated by estimating the embrittlement quantity of the evaluation-target member from the operation data. In addition, it is possible to respond to changes in the use temperature due to load variation or the like of power generator by sequentially calculating the evaluation-target site temperature from the operation data and using the calculated result for the embrittlement evaluation. Furthermore, in the data storage unit, which stores various statistics used for the embrittlement evaluation and experimentally calculated constants or the like included in the embrittlement prediction formulas, the stored data can be updated based on data obtained from outside the embrittlement estimation device, so that newly obtained embrittlement measurement data can be used for future embrittlement evaluation.

### Configuration of Second Embodiment

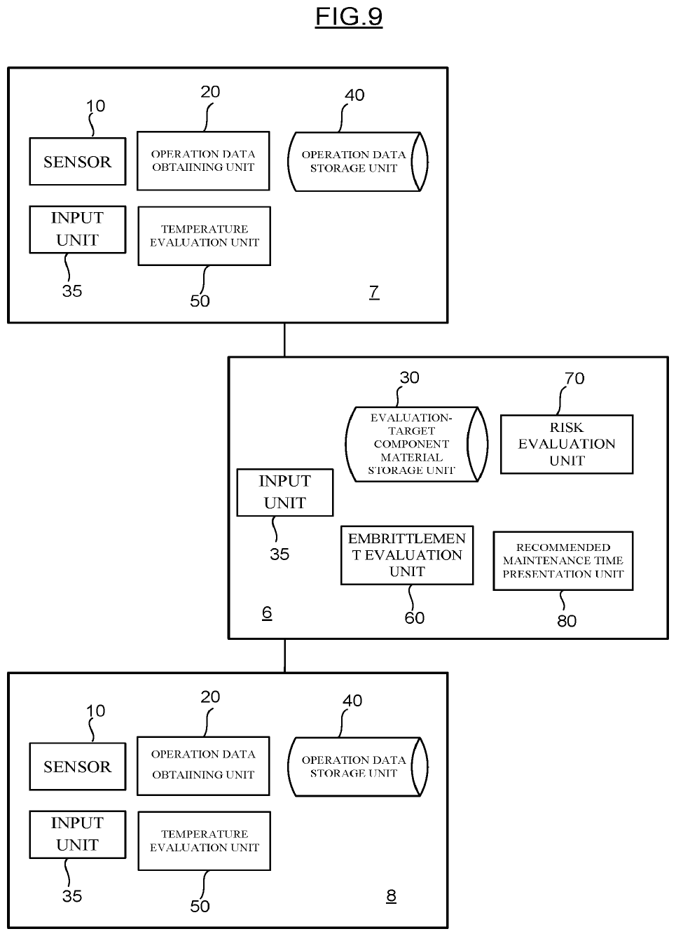

A second embodiment configures a device using a module including an evaluation unit and a storage unit, which configures the material deterioration evaluation device 1 presented as the first embodiment, and an embrittlement evaluation module including an embrittlement evaluation unit. FIG. 9 illustrates a material deterioration evaluation device configured by combining two modules and an embrittlement evaluation module.

An embrittlement evaluation module 6, including the embrittlement evaluation unit 60, has the input unit 35, the evaluation-target component material storage unit 30, the embrittlement evaluation unit 60, the risk evaluation unit 70, and the recommended maintenance time presentation unit 80. Modules 7 and 8, which are used in combination with the module 6, each have the sensor 10, the operation data obtaining unit 20, the input unit 35, the operation data storage unit 40, and the temperature evaluation unit 50.

The modules 7, 8 perform everything from the operation data obtaining to the historical data creation and the calculation of the evaluation-target site temperature. As in the first embodiment, the operation data are collected from the sensor 10 in the operation data obtaining unit 20 and saved in the operation data storage unit 40. The temperature evaluation unit 50 calculates the evaluation-target site temperature using the operation data or historical data and the calculated temperature is saved in the operation data storage unit 40.

The embrittlement evaluation module 6 performs everything from the embrittlement evaluation to the recommendation of the maintenance time. The embrittlement evaluation module 6 collects the evaluation-target site temperature evaluated by the modules 7, 8 and the historical data of operation. Based on these data, embrittlement is evaluated by the embrittlement evaluation unit 60 and the result is stored in the evaluation-target component material storage unit 30, as in the first embodiment. The risk evaluation unit 70 evaluates the risk, and the recommended maintenance time presentation unit 80 presents the recommended maintenance time based on the embrittlement evaluation results, risk evaluation results, operation plan, and other data. The recommended maintenance time presented by the recommended maintenance time presentation unit 80 may be determined by evaluating the damage risk or the like to the evaluation-target site of each module individually, or by considering a mutual state of each device to align or stagger the recommended maintenance time while considering the evaluation results of each device, the operating rate and maintainability of the equipment to be evaluated, and other data.

The embrittlement evaluation module 6 is connected to the modules 7, 8 by a wired or wireless connection capable of data communication. Alternatively, data may be exchanged using various media such as memory cards. Various terminals necessary for data communication are included in the input unit 35. Connection and data communication with auxiliary devices may be made at all times or after a lapse of a certain time. The time may be determined in advance or based on the operation data.

The sensor 10, the operation data obtaining unit 20, the input unit 35, the operation data storage unit 40, and the temperature evaluation unit 50 are included in each of the modules 7, 8 in the above example, but the device configuration is not limited thereto. The configuration can be changed, such as including the temperature evaluation unit 50 in the embrittlement evaluation module 6. Also, depending on the device configuration, the above-mentioned operation data storage unit 40 or evaluation-target component material storage unit 30 can be divided into multiple units, and the data storage unit can be included in both the embrittlement evaluation module 6 and module 7 or 8.

For example, the operation data storage unit 40 saves the operation data obtained by the operation data obtaining unit 20, the temperature data calculated by the temperature evaluation unit 50 based on the operation data, and the evaluation formulas used by the temperature evaluation unit 50. That is, when the temperature evaluation unit 50 is included in the embrittlement evaluation module 6, the device may be configured such that the operation data storage unit 40 is also provided in each of the embrittlement evaluation module 6 and the module 7 or 8. The operation data storage unit 40 included in the module 7 or 8 saves the operation data obtained by the operation data obtaining unit 20, and the operation data storage unit 40 included in the embrittlement evaluation module 6 saves the temperature data calculated by the temperature evaluation unit 50 and the evaluation formulas used for the calculation. Although the example of connecting two modules to the embrittlement evaluation module 6 is presented, the number of modules that can be connected to the embrittlement evaluation module is not limited to two, but may be increased to more than two, or may be one.

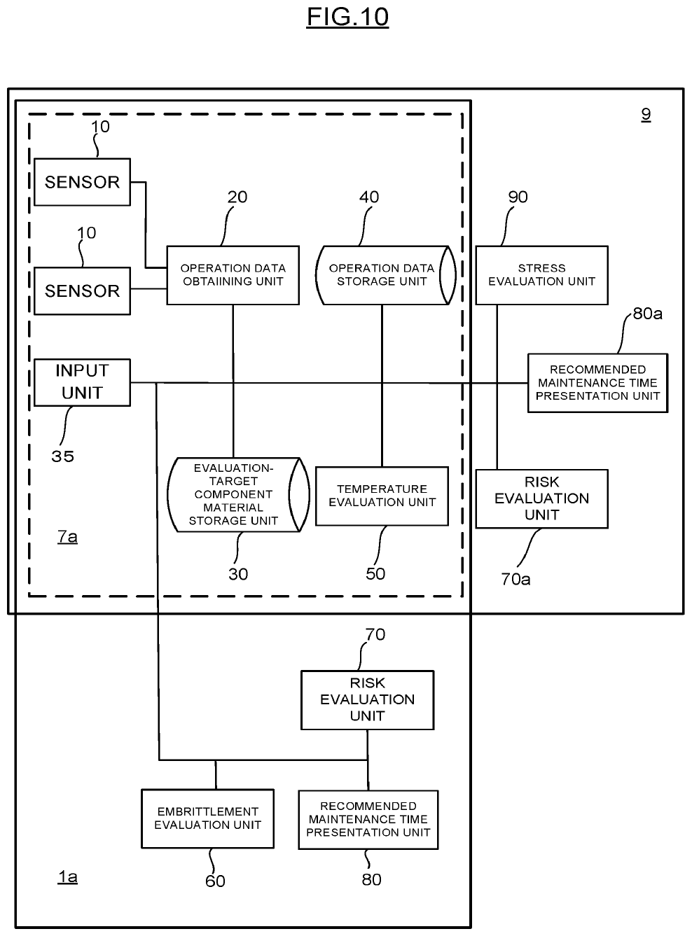

Various sensors, each evaluation unit, and data storage unit included in the modules of the material deterioration evaluation device may be shared with sensors, evaluation units, and data storage units that configure other material deterioration evaluation devices and damage evaluation devices, including the damage evaluation device mentioned above. FIG. 10 illustrates a shared example of modules that configure the material deterioration evaluation device. In FIG. 10, a numeral 9 is the aforementioned damage evaluation device, which evaluates the evaluation-target site temperature and generated stress based on the operation data of the equipment to be evaluated, and evaluates fatigue damage, creep damage, or crack growth damage caused by the damages. The damage evaluation device 9 includes the sensors 10, the operation data obtaining unit 20, the evaluation-target component material storage unit 30, the input unit 35, the operation data storage unit 40, the temperature evaluation unit 50, a stress evaluation unit 90, a risk evaluation unit 70a, and a recommended maintenance time presentation unit 80a.

As in the first embodiment, the operation data obtaining unit 20 samples temperature, pressure, strain, equipment power, and other data obtained by the sensors 10 installed in members configuring the equipment to be evaluated as the operation data, and stores the data in the operation data storage unit 40.

The temperature evaluation unit 50 calculates the evaluation-target site temperature using the operation data saved in the operation data storage unit 40. When calculating the evaluation-target site temperature, a relational formula between the equipment power or the measured value of any sensor 10 and the evaluation-target site temperature is previously stored in the operation data storage unit 40, and the evaluation-target site temperature is calculated by using the relational formula. The calculated temperature data is saved in the operation data storage unit 40 in association with the operation data.

The stress evaluation unit 90 calculates stress generated at the evaluation-target site using the operation data saved in the operation data storage unit 40. To calculate the stress, a relational formula between the equipment power or the measured values of any of the sensors 10 and the generated stress is previously saved in the operation data storage unit 40, and the stress generated at the evaluation-target site is calculated using the relational formula. The calculated stress is saved in the operation data storage unit 40 in association with the operation data.

The risk evaluation unit 70a calculates the fatigue damage and the creep damage quantities from the operation data stored in the operation data storage unit 40, the temperature associated with the operation data, the stress evaluation result, and the material data such as fatigue strength properties and creep properties of the evaluation-target site. The damage risk is evaluated based on the calculated damage quantity and operation data. The calculated damage quantity and damage risk evaluation result are stored in the evaluation-target component material storage unit 30.

The recommended maintenance time presentation unit 80a predicts the damage quantity in the future based on the damage evaluation results stored in the evaluation-target component material storage unit 30, the operation data stored in the operation data storage unit 40, and the future equipment operation plan that is separately input and presents the recommended maintenance time.

When the site to be evaluated of a material deterioration evaluation device 1a and the site to be evaluated of the damage evaluation device 9 are the same, or when the temperatures of both sites to be evaluated can be calculated by the temperature evaluation unit 50, the functions of both devices can be fulfilled by sharing the sensors, evaluation unit, and other units that configure the devices. The sensors 10, the operation data obtaining unit 20, the evaluation-target component material storage unit 30, the input unit 35, the operation data storage unit 40, and the temperature evaluation unit 50, which configure the material deterioration evaluation device 1a can be made into a module 7a, which can be shared with the damage evaluation device 9 to configure the device.

In the above example, the sensors 10, the operation data obtaining unit 20, the evaluation-target component material storage unit 30, the input unit 35, the operation data storage unit 40, and the temperature evaluation unit 50 are made into a module, but the configuration to be included in the module is not limited to the above and can be changed. In addition, the module is shared by two devices, the damage evaluation device 9 and the material deterioration evaluation device 1a in the example, but the number of devices that can share the module is not limited thereto, and the number of devices can be increased. The above is the second embodiment.

While certain embodiments have been described, these embodiments have been presented by way of example only, and are not intended to limit the scope of the inventions. Indeed, those novel embodiments may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

### REFERENCE SIGNS LIST

1, 1a . . . material deterioration evaluation device, 2 . . . turbine equipment, 3 . . . turbine casing, 4 . . . rotor, 5 . . . rotor blade, 6 . . . embrittlement evaluation module, 7, 7a, 8 . . . module, 9 . . . damage evaluation device, 10 . . . sensor, 11 . . . steam inlet, 12 . . . steam outlet, 13 . . . steam inlet, 14 . . . steam outlet, 20 . . . operation data obtaining unit, 30 . . . evaluation-target component material storage unit, 40 . . . operation data storage unit, 50 . . . temperature evaluation unit, 60 . . . embrittlement evaluation unit, 70, 70a . . . risk evaluation unit, and 80, 80a . . . recommended maintenance time presentation unit.

1. A material deterioration evaluation device for evaluating embrittlement of equipment, comprising:
an operation data obtaining unit for detecting and obtaining a state of the equipment as operation data;
an operation data storage unit for saving the operation data;
a temperature evaluation unit for calculating a predetermined evaluation-target site temperature of the equipment based on the operation data;
an evaluation-target component material storage unit for storing material data of a material forming the equipment and embrittlement estimation formulas;
an embrittlement evaluation unit for calculating an embrittlement quantity of the material forming the equipment based on the evaluation-target site temperature, the material data, and the embrittlement estimation formulas;
a risk evaluation unit for calculating a damage risk of the material that forms the equipment based on the embrittlement quantity; and
a recommended maintenance time presentation unit for presenting a recommended maintenance time of the equipment based on the damage risk.

2. The material deterioration evaluation device according to claim 1, wherein
a plurality of modules each including at least one unit from among the operation data obtaining unit, the operation data storage unit, the temperature evaluation unit, the evaluation-target component material storage unit, the embrittlement evaluation unit, the risk evaluation unit, and the recommended maintenance time presentation unit are included, and
the plurality of modules are capable of communicating data among the plurality of modules.

3. The material deterioration evaluation device according to claim 1, wherein
the risk evaluation unit calculates the damage risk using statistics on errors in the embrittlement estimation formulas stored in the evaluation-target component material storage unit.

4. The material deterioration evaluation device according to claim 3, wherein
the statistics on the errors in the embrittlement estimation formulas stored in the evaluation-target component material storage unit are rewritable.

5. A material deterioration evaluation method for evaluating an embrittlement quantity of equipment, comprising:
obtaining the embrittlement quantity as a function of a saturated embrittlement quantity and time; and
calculating the saturated embrittlement quantity of an evaluation-target site used for the evaluation of the embrittlement quantity by multiplying
a constant (A1) that is experimentally determined in advance,
a constant (B) that is calculated from quantities of elements contained in a material forming the equipment, and
an exponential function whose exponent is a product of an inverse of a linear function of temperature and a constant (A2) that is experimentally determined in advance.

6. The material deterioration evaluation method according to claim 5, wherein
the exponential function is an exponential function of a base of a natural logarithm.

7. The material deterioration evaluation method according to claim 5, wherein
the saturated embrittlement quantity of the evaluation-target site used for the evaluation of the embrittlement quantity is obtained by the following formula:
saturated embrittlement quantity=A1×B×exp{A2/(K)}
where A1 and A2 are constants obtained experimentally in advance, B is a constant calculated from the quantities of elements contained in the material forming the equipment, and K is an absolute temperature.

8. The material deterioration evaluation method according to claim 5, wherein
the constant (B) calculated from the quantities of elements contained in the material forming the equipment is calculated from any one of or a plurality of quantities of elements from among eight elements of P, Si, Mn, Cu, Ni, Sn, Sb, and As.

9. The material deterioration evaluation device according to claim 1, wherein
the embrittlement evaluation unit evaluates the embrittlement by
obtaining the embrittlement quantity as a function of a saturated embrittlement quantity and time; and
calculating the saturated embrittlement quantity of an evaluation-target site used for the evaluation of the embrittlement quantity by multiplying
a constant (A1) that is experimentally determined in advance,
a constant (B) that is calculated from quantities of elements contained in a material forming the equipment, and
an exponential function whose exponent is a product of an inverse of a linear function of temperature and a constant (A2) that is experimentally determined in advance.

10. The material deterioration evaluation device according to claim 9, wherein
an embrittlement quantity per unit time is calculated in advance, and
the embrittlement quantity is calculated by adding the embrittlement quantity per unit time based on a time over which the evaluation-target site temperature can be considered constant.

11. The material deterioration evaluation device according to claim 10, wherein
the previously and experimentally determined constants and the embrittlement quantity per unit time are stored in the evaluation-target component material storage unit and are rewritable.

12. The material deterioration evaluation device according to claim 9, wherein
the constant (B), which is calculated from the quantities of elements contained in the material forming the equipment is back-calculated using the embrittlement quantity of the material forming the equipment at a certain time and the operation data stored in the operation data storage unit, and the embrittlement quantity is evaluated by using the back-calculated constant.
